(19) United States
(12) Plant Patent Application Publication
(10) Pub. No.: US 2003/0126656 P1
(43) Pub. Date: Jul. 3, 2003

(54) NEMESIA PLANT NAMED 'PENVAL'

(76) Inventor: Sidney James Jones, St. Bridges (GB)

Correspondence Address: PLANTHAVEN, INC, GEOFFREY NEEDHAM, 121 W. PUEBLO St., STE 14, P.O. Box 3056, SANTA BARBARA, CA, 93130-3056, US

(21) Appl. No.: 10/035,304

(22) Filed: Dec. 28, 2001

(51) Int. Cl. A01H 5/00

(52) U.S. Cl. PLT/263

(57) ABSTRACT

A new and distinct cultivar of Nemesia plant named ‘Penval’ that is characterized by a dense, compact habit, narrow green leaves, upright flower spikes and a floriferous display of pink-purple flowers. In combination these characteristics set ‘Penval’ apart from all other existing varieties of Nemesia known to the inventor.

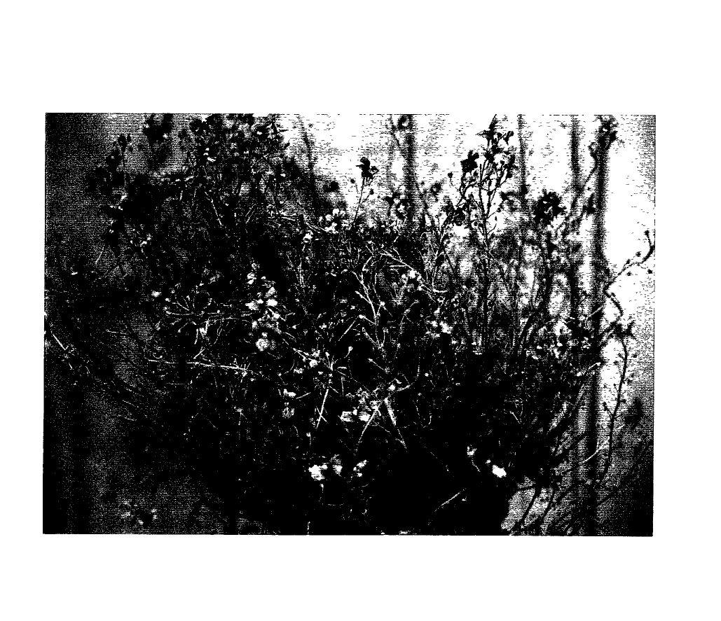

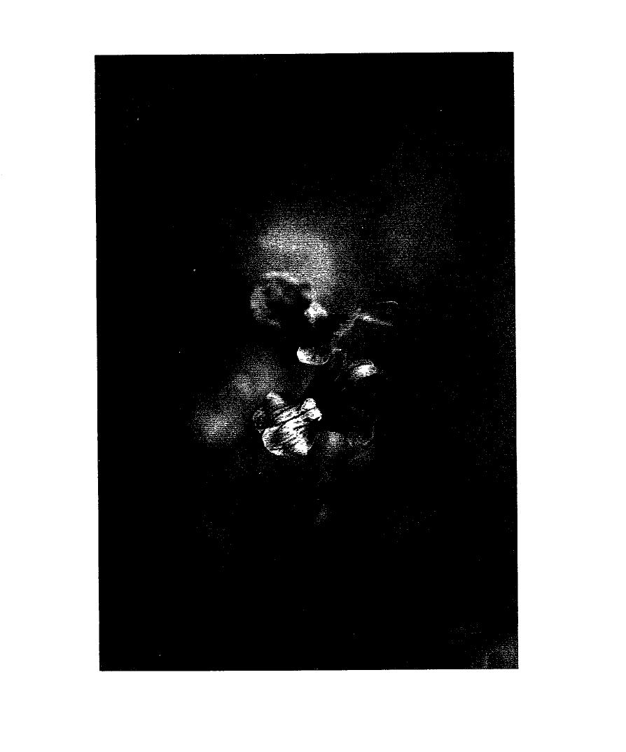

### BACKGROUND OF THE INVENTION

The present invention relates to a new and distinct cultivar of Nemesia plant that will be referred to hereinafter by the cultivar name ‘Penval’. ‘Penval’ is a hybrid that resulted from a long term breeding program, involving cross-pollination of unidentified Nemesia hybrids, and was selected by the inventor in a cultivated area of Netherwent, Wales in 1996. The female parent is an unidentified Nemesia species hybrid and the male parent is an unidentified Nemesia species hybrid.

The breeding program began eight years ago with the objectives of improving upon such well-known varieties of Nemesia as ‘Confetti’, ‘Innocence’ and ‘Joan Wilder’. The inventor sought to first improve on habit by breeding plants that are more compact with rigid, upright flower stalks. Secondly the inventor sought to increase the size of the flower and the length of flowering time, and thirdly to improve the color range by producing clear colors. The fourth objective was to convert the species to a more efficient vegetative propagation rather than rely on seed, which is inherently unstable.

In 1995 the inventor made intervarietal and interspecific crosses with such common varieties as ‘Confetti’, ‘Innocence’, ‘Joan Wilder’, ‘Woodcote’ (all of these are unpatented), and identified seedlings from a cultivated area of Netherwent, Wales, that had resulted from uncontrolled crosses. The inventor selected from the flowering plants those that showed increasing expression of the desired traits. Re-selection, back-crossing and new crossing was conducted to produce parent lines which could then be crossed to produce the final product.

‘Penval’ is distinguished from all other Nemesia known to the inventor by its combination of dense compact growth, narrowness of leaf, rigid upright flower spikes, number of flower spikes, flower size, and flower color in this color category.

‘Penval’ was first asexually propagated by the inventor in 1997 in Netherwent, Wales using vegetative cuttings. It has been found stable and true to type after many cycles of vegetative cuttings.

### SUMMARY OF THE INVENTION

The following traits have been repeatedly observed and represent the distinguishing characteristics of ‘Penval’. In combinaton these traits set the new cultivar apart from all other existing varieties of Nemesia known to the inventor. ‘Penval’ has not been tested under all possible conditions and phenotypic differences may be observed with variations in environmental, climatic, and cultural conditions, however, without any variance in genotype.

1. Nemesia ‘Penval’ exhibits a dense, compact habit.

2. Nemesia ‘Penval’ flowers profusely and exhibits fragrant pink-purple flowers.

3. Nemesia ‘Penval’ exhibits an abundance of flower spikes, unique colored flowers and large flowers for the pink color category of Nemesia.

4. Nemesia ‘Penval’ exhibits rigid, upright flower spikes.

5. Nemesia ‘Penval’ exhibits narrow green leaves.

6. Nemesia ‘Penval’ is hardy to minus 5° Centigrade.

### BRIEF DESCRIPTION OF THE DRAWINGS

The accompanying drawings illustrate the distinguishing traits and overall appearance of the new Nemesia cultivar ‘Penval’. Drawings were made of plants that are 12 months old and grown in two-gallon containers under greenhouse conditions in Encinitas, Calif. Sheet 1 illustrates the entire plant and habit from a side perspective. Sheet 2 is a close-up view of the flower. The drawings were made using conventional photographic techniques and although colors may appear different from actual colors due to light reflectance, they are as accurate as possible by conventional photography and copying techniques.

### BOTANICAL DESCRIPTION OF THE PLANT

The following is a detailed description of the Nemesia plant named ‘Penval’. Data was collected in Arroyo Grande, Calif. from plants grown in two-gallon containers under greenhouse conditions. Phenotypic differences may be observed with variations in environmental, climatic, and cultural conditions. The color determinations are in accordance with The Royal Horticultural Society Colour Chart except where general color terms of ordinary dictionary significance are used. Growing requirements are similar to other Nemesia plants.

Botanical classification: Nemesia ‘Penval’.

Common name: Nemesia.

Use: Bedding and patio plant.

Parentage: The parents of Nemesia ‘Penval’ are unidentified Nemesia hybrids.

Female parent.—Unidentified Nemesia species hybrid.

Male parent.—Unidentified Nemesia species hybrid.

Propagation: Vegetative cuttings.

Sexuality: Hermaphrodite.

Growth Habit: Dense, compact habit.

Plant dimensions: 30 cm. in height and 50 cm. in width in a 4-inch container.

Type: Hardy perennial.

Time to initiate roots: Approximately 16 days at temperatures of 21° Centigrade are needed to produce rooted cuttings.

Crop time: 12 months are required to produce a finished two-gallon from a rooted cutting.

Root system: Numerous and fine.

Hardiness: Hardy to minus 5° Centigrade.

Stem:

Stem shape.—Quadrilateral.

Stem length.—38 cm. in length.

Stem diameter.—2 mm. in diameter.

Stem surface.—Stipitate glandular.

Stem color.—138B.

Stem texture.—Flexible with corner ridges and occasional flexuous stems.

Internode length.—2-8 cm. between nodes.

Branching habit.—Ascending and freely branching.

Foliage:

Leaf shape.—Lanceolate.

Leaf division.—Simple.

Apex.—Acute.

Base.—Rounded.

Margins.—Closest to crenate.

Surface.—Glabrous.

Arrangement.—Opposite.

Leaf length.—2 cm. in length.

Leaf width.—1 cm. in width.

Leaf color (adaxial surface).—138A.

Leaf color (abaxial surface).—138B.

Petiole dimensions.—1 mm. in length and less than 0.50 mm. in diameter.

Vein pattern.—Prominent mid-vein depressed on adaxial surface and protruding on abaxial surface.

Flowers:

Flowering season.—Spring and summer.

Fragrance.—Sweet scent.

Self-cleaning or persistent.—Self-cleaning.

Inflorescence dimensions.—8 cm. in length and 3 cm. in width.

Type of inflorescence.—Terminal racemes.

Quantity of flowers.—Floriferous. Approximately 15 flowers per primary peduncle.

Primary peduncle dimensions.—12 cm. in length and 1 mm. in diameter.

Secondary peduncle dimensions.—6 mm. in length and less than 1 mm. in width.

Peduncle surface.—Stipitate glandular.

Peduncle color.—138B.

Bud shape.—Oval.

Bud color.—155A.

Bud dimensions.—3 mm. in diameter and 5 mm. in length.

Flower shape.—Personate.

Palate color.—16A.

Nectary color.—163A.

Nectary dimensions.—3 mm. in width and 2 mm. in length.

Palate dimensions.—3 mm. in length and 2 mm. in width.

Surface.—Glabrous.

Petal margin.—Entire.

Petals.—Four in number.

Petal color (Upper lip).—78C.

Petal color (lower lip).—78B.

Throat color (inside).—89D.

Dark markings on flower.—89D.

Petal dimensions (upper three).—1.5 cm. in width and 1 cm. in length.

Petal dimensions (lower petal).—8 mm. in width and 6 mm. in length.

Flower spur dimensions.—6 mm. in length and 1 mm. in diameter.

Spur color.—1D.

Calyx dimensions.—3 mm. in height and 4 mm. in width.

Sepals.—Five in number.

Sepal surface.—Stipitate glandular.

Sepal apex.—Acute.

Sepal color.—138A.

Reproductive organs:

Stamens.—Four in number and didynamous.

Color of stamens.—155A.

Stamen dimensions.—1 mm. in length and less than 0.25 mm. in diameter.

Anther color.—8B.

Anther dimensions.—Less than 0.50 mm. in length and less than 0.50 mm. in width.

Pistil.—One

Pistil color.—155A.

Pistil dimensions.—0.50 mm. in length and 0.50 mm. in diameter.

Ovary dimensions.—1 mm. in height and 2 mm. in width.

Ovary shape.—Oval.

Ovary position.—Superior.

Ovary color.—145B.

Seed production:

Quantity of seed.—Approximately 15 fertile seeds per capsule.

Capsules.—Approximately 20 capsules per flowering spike, but this is markedly influenced by how many times the plant is pinched back.

Capsule dimensions.—12 mm. in length and 7 mm. in width.

Capsule color.—177D.

Capsule surface.—Glossy.

Appearance of seed.—Flattened and winged.

Seed color.—200D and wing 156D.

Shape of seed.—Oval.

Seed dimensions.—3 mm. in length and 2.5 mm. in width.

1. A new and distinct cultivar of Nemesia plant named ‘Penval’ as described and illustrated herein.
